(19) United States
(12) Patent Application Publication
(10) Pub. No.: US 2013/0244905 A1
(43) Pub. Date: Sep. 19, 2013

(54) Reporter for RNA Polymerase II Termination

(71) Applicants: Ed Grabczyk, New Orleans, LA (US); Mimi C. Sammarco, New Orleans, LA (US)

(72) Inventors: Ed Grabczyk, New Orleans, LA (US); Mimi C. Sammarco, New Orleans, LA (US)

(21) Appl. No.: 13/807,959

(22) PCT Filed: Jun. 24, 2011
(86) PCT No.: PCT/US2011/041815

§ 371 Date: May 20, 2013

Related U.S. Application Data
(60) Provisional application No. 61/361,710, filed on Jul. 6, 2010.

(51) Int. Cl. C12Q 1/68

(52) U.S. Cl. CPC C12Q 1/6897
USPC 506/10; 435/320.1; 435/325; 435/6.13

(57) ABSTRACT

A “tandem” reporter construct is disclosed that is capable of assaying RNA transcription termination. The ratio of expression between an upstream reporter and a downstream reporter as compared to the ratio observed for a control construct provides a measure of the relative rate of successful elongation through the intervening sequence. In one embodiment, two self-cleaving ribozymes separate the reporters from a test sequence between them.

The benefit of the 6 Jul. 2010 filing date of U.S. provisional patent application Ser. No. 61/361,710 is claimed under 35 U.S.C. §119(e) in the United States, and is claimed under applicable treaties and conventions in all countries.

This invention was made with support from the United States Government under grant R01NS046567 awarded by the National Institutes of Health. The United States Government has certain rights in this invention.

### TECHNICAL FIELD

This invention pertains to compositions and methods for measuring RNA transcription elongation and termination, whether in vitro or in vivo.

### BACKGROUND ART

It is unknown precisely what percentage of human RNA polymerases initiate and successfully complete gene transcription; live cell imaging has given estimates that are on the order of 1%. The RNA polymerase II transcription complex (RNAP II) may be influenced by factors that modify the transcription complex, by factors that modifying the chromatin, or by intrinsic properties of the template and transcript that affect the dynamic balance between transcription elongation and transcription termination. The early termination of transcription usually occurs near the promoter. Once RNA polymerase II progresses past the promoter region, it shifts into a highly processive mode in which it can continue active transcription for megabases.

An elongating polymerase must maintain a balance between terminating too readily, such that it does not reach the end of a gene, and being an unstoppable juggernaut that continues transcription past the end of a gene. When an RNAP II molecule engages in nonproductive elongation, it not only wastes energy but it also risks interfering with the expression of neighboring genes. More importantly, evidence is accumulating that termination is required for efficient mRNA processing and export, and thus for proper protein expression. Proper termination has been linked to mRNA 3′-end processing and mRNA export through the nuclear pore in yeast cells. Proper transcription termination is likely to be linked to proper mRNA processing and optimal gene expression in human cells as well. Consequently, multiple mechanisms have evolved to terminate transcription efficiently at the ends of genes. Transcription termination is an important step in gene expression, but it can be difficult to measure experimentally, especially in mammalian cells.

Three models have been proposed for polyadenylation-associated transcription termination: the “allosteric” model, the “torpedo” model, and the combined allosteric/torpedo model. The original formulation of the allosteric, or anti-terminator model, posited that polyadenylation signals cause a change in the complement of various anti-termination factors that are associated with RNAP II, which render the complex more susceptible to termination. An early formulation of the torpedo model suggested that a 5′-to-3′ exonuclease attacked the transcript at the poly(A) cleavage point, and degraded the nascent transcript in the direction towards the RNA polymerase, thereby leading to termination. More recently, there has been evidence for a combined allosteric/torpedo model. It has also been suggested that the phosphorylation state of the RNAP II C-terminal domain may play a role in termination and 3′ end formation.

Transcription termination is an important, yet under-studied aspect of gene regulation. In many cases, transcription elongation is the rate-limiting step in gene expression. The “classic” technique used in research on transcription elongation and termination has been the nuclear run-on assay (NRO): RNAP II complexes in prepared nuclei incorporate a radiolabeled rNTP as they “run-on” for some distance during incubation in vitro. Radiolabeled RNA is then hybridized to one or more specific targets to assess relative levels of active RNA transcription at the time the nuclei were prepared. However, the NRO assay is cumbersome and is not especially sensitive. The NRO assay requires about 2×10⁷ cells per sample and a strong promoter to work. Most single copy mammalian genes do not have expression levels sufficient to produce reliable signals for an NRO assay. Consequently, much NRO analysis has been performed using transiently transfected plasmids, rather than native genes in native chromatin. While a great deal can be learned from transient assays, such transient assays can obscure the important role in termination played by factors such as epigenetic chromatin modification. Conversely, chromatin immunoprecipitation (ChIP) techniques using anti-RNAP II antibodies can show the association of RNAP II with a particular DNA region within native chromatin, the ChIP technique cannot determine whether the polymerase was actively transcribing.

The “torpedo” model of transcription termination predicts that an exonuclease or helicase enters the nascent transcript at the poly(A) cleavage point and contributes to termination. A 5′-to-3′ exonuclease (Rat1p in yeast, Xrn2 in humans) contributes to RNAP II termination and 3′ end formation. The “torpedo” model proposes that Xrn2 loads either onto the nascent transcript at the poly(A) cleavage site, or onto the free end generated by CoTC cleavage in the case of HBB termination, and then degrades the nascent transcript, eventually catching up to the RNA polymerase, and causing it to release from the template. The helicase senataxin (or sen1p in yeast) may also play a role in transcription termination. The yeast homologue Rat1p prefers RNA with a 5′ monophosphate as a substrate, which may explain why intact, capped messages are not a target. Furthermore, Rat1p activity on substrates with a 5′ OH or a structured 5′ end is also greatly reduced relative to that for the preferred substrate.

E. Grabczyk et al., “The GAA•TTC triplet repeat expanded in Friedrich's ataxia impedes transcription elongation by T7 RNA polymerase in a length and supercoil dependent manner,” Nucl. Acids Res., vol. 28, pp. 2815-2822 (2000) reported that large expansions of the trinucleotide repeat GAA•TTC within an intron caused Friedrich's ataxia, as a result of the adverse effect that the GAA•TTC tract had upon mRNA transcription. Constructs incorporating a self-cleaving ribozyme sequence were used to facilitate comparison of transcription products from both long, linear and supercoiled templates.

E. Grabczyk et al., “A persistent RNA•DNA hybrid formed by transcription of the Friedrich ataxia triplet repeat in live bacteria, and by T7 RNAP in vitro,” Nucl. Acids Res., vol. 35, pp. 5351-5359 (2007) reported that the expansion of an unstable GAA•TTC repeat within an intron causes Friedrich's ataxia by reducing expression of frataxin. It was reported that transcription causes extensive RNA•DNA hybrid formation on GAA•TTC templates. It was hypothesized that the RNA•DNA hybrids played a role in GAA•TTC tract instability. Constructs incorporating a self-cleaving ribozyme sequence were used to obtain defined transcripts from a supercoiled template.

J. Ditlevson et al., “Inhibitory effect of a short Z-DNA forming sequence on transcription elongation by T7 RNA polymerase,” Nucl. Acids Res., vol. 36, pp. 3163-3170 (2008) reported that the Z-DNA forming sequence (CG)₁₄ caused genomic instability in both mammalian cells and bacteria, and that this effect increased with transcription. Partial transcription blockage was detected. A self-cleaving ribozyme sequence was used to assist in determining the probability of transcription blockage.

A. Teixera et al., “Autocatalytic RNA cleavage in the human β-globin pre-mRNA promotes transcription termination,” Nature, vol. 432, pp. 526-530 (2004) reported that co-transcriptional self-cleavage within beta-globin pre-mRNA was critical for transcription termination. Transcription termination was determined by nuclear run-on analysis.

N. Gromak et al., “Pause sites promote transcriptional termination of mammalian RNA polymerase II,” Mol. Cell. Biol., vol. 26, pp. 3986-3996 (2006) reported that certain pause elements play a role in transcription termination. Transcription termination was determined using nuclear run-on probes.

S. West et al., “Molecular dissection of mammalian RNA polymerase II transcriptional termination,” Molecular Cell, vol. 29, pp. 600-610 (2008) reported studies on the timing and relationship of RNA transcript release and DNA template release in transcriptional termination. The studies employed nuclear run-on analysis.

See also M. Sammarco, The iron chef today's mystery ingredient, Frataxin, PhD Thesis, Louisiana State University Health Sciences Center (New Orleans, La., 2005).

There is an unfilled need for improved techniques to measure RNA transcription termination, especially in mammalian cells.

### DISCLOSURE OF THE INVENTION

We have discovered a novel “tandem” reporter construct that is capable of assaying transcription termination when a single copy of the construct is integrated into a chromosome, including a human or other mammalian chromosome. Alternatively, the construct may be used as a multicopy episome, or a multicopy chromosomal insert. The ratio of expression between a downstream reporter as compared to that for a control reporter upstream provides a measure of the relative rate of successful elongation through the intervening sequence.

In a prototype example, the novel construct was used to measure the efficiency of termination within fragments of the human beta-actin (ACTB) and human beta-globin (HBB) terminator regions. The novel system provided a sensitive, ratiometric measure of transcription termination in live cells, compatible with high throughput approaches.

We also used the novel construct to explore the relative contributions of the torpedo model versus the allosteric model of transcription termination. Our observations did not support a dominant role for either model, nor for any other single factor tested such as XRN2 or Senataxin, in termination mediated by HBB or ACTB sequences. Rather, our findings suggest instead that multiple elements cooperatively contribute to termination, with efficiency mediated by redundancy.

The novel tandem reporter construct is capable of assaying transcription termination when the construct is present in a cell either as an extra-chromosomal episome (e.g., a multi-copy plasmid), or when several copies or preferably a single copy of the construct is integrated into a chromosome, whether a human chromosome, other mammalian chromosome, or other eukaryotic or prokaryotic chromosome. The use of a single copy of the construct integrated into a chromosome will, in many cases, otherwise most closely approximate the behavior of a native gene. The ratio of expression between a downstream reporter as compared to that from an upstream control reporter provides a measure of the relative rates of successful elongation through the intervening sequence. Two self-cleaving ribozymes separate the reporters from a test sequence between them.

In lieu of the preferred self-cleaving ribozyme sequence(s), alternatively another target sequence may be used that is recognized by a cleaving ribozyme, protein or complex. For example, there are (non-self-cleaving) ribozymes that target and cleave RNA, and there are proteins that can cleave RNA with some sequence specificity—although in the latter case the specificity is typically not high. As another alternative, RNA may be targeted for cleavage with the combination of a complementary DNA oligomer and an RNase H enzyme. (RNase H specifically cleaves the RNA in an RNA•DNA hybrid). However, these bimolecular and higher order interactions are not preferred due to their generally lower efficiencies both in the speed and completeness of cleavage. By contrast, the intramolecular reaction that is catalyzed by an efficient self-cleaving ribozyme can occur almost immediately after the ribozyme is transcribed, with an efficiency approaching 100%. Also, while a self-cleaving ribozyme cleaves only itself, these other alternatives are likely to result in non-target cleavages of other sequences.

Several types of self-cleaving ribozymes are known in the art. Generally, any efficient, self-cleaving ribozyme may be used in practicing this invention. Examples of types of self-cleaving ribozymes include the hammerhead, hairpin, glmS, hepatitis delta virus (HDV) and Varkud satellite (VS) ribozyme types.

To the inventors' knowledge, no prior system has been able to successfully distinguish between whether effects on downstream expression actually reflected termination of transcription, versus whether those effects might instead have resulted from cleavage of the transcript, for example at a poly(A) site. The novel reporter system overcomes this problem by using self-cleaving ribozymes to separate the effects of RNA processing from the effects of transcription. The novel system is highly effective at detecting termination. The novel system is useful, for example, in high-throughput assays for identifying transcription terminators and for measuring terminator efficiency. In prototype experiments we have used the system to measure the efficiency of termination as influenced by multiple elements contained within fragments of the human beta-actin (ACTB) and human beta-globin (HBB) terminator regions. The novel system provided a sensitive measure of transcription termination in living cells. Size matched fragments containing the polyadenylation signal of the human beta-actin gene (ACTB) and the human beta-globin gene (HBB) were evaluated for transcription termination using this new ratiometric tandem reporter assay. Constructs bearing just 200 base pairs on either side of the consensus poly(A) addition site terminated 98% and 86% of transcription for ACTB and HBB sequences, respectively. The nearly 10-fold difference in read-through transcription between the two short poly(A) regions was eclipsed when additional downstream poly(A) sequence was included for each gene. Both poly(A) regions proved very effective at termination when 1100 base pairs were included, stopping 99.6% of transcription. To determine if part of the increased termination was simply due to the increased template length, we inserted several kilobases of heterologous coding sequence downstream of each poly(A) region test fragment. Unexpectedly, the additional length reduced the effectiveness of termination of HBB sequences 2-fold and of ACTB sequences 3- to 5-fold.

The tandem construct provides a sensitive measure of transcription termination in human cells. Decreased Xrn2 or Senataxin levels produced only a modest release from termination. Our data support overlap in allosteric and torpedo mechanisms of transcription termination and suggest that efficient termination is ensured by redundancy.

The ratiometric measurements that may be obtained with the novel construct are well suited for high-throughput screening applications. The ratiometric measurement is essentially self-normalizing for every cell. Consequently, it is independent of cell number, which helps to reduce errors that might otherwise be introduced by variations in cell number, or variations between wells containing cell samples. Because both reporters are contained in the same vector, the ratiometric measure is independent of vector copy number, and is therefore also well-suited for use in multi-copy vector approaches, which could otherwise be considered undesirable for high-throughput screening techniques.

A Tandem Reporter System Quantifies Transcription Elongation

The novel system uses a tandem reporter construct to measure the rate of transcription termination in living cells. Two quantifiable reporters are expressed in tandem from a transcriptional promoter, preferably an inducible promoter, in a construct that may be located episomally or integrated into a chromosome, and present either in a single copy or in multiple copies. Preferably, the construct is located at a single, unique chromosomal location. In a prototype embodiment, for example, transcription initiates at a tetracycline-inducible promoter, proceeds through the first reporter (FLUC), and then must traverse a test termination sequence (or “linker”) before reaching a second reporter (hRLUC).

FIG. 1A depicts schematically a prototype embodiment of the invention. A tetracycline-inducible promoter drives transcription through the two tandem reporters. Two self-cleaving hammerhead ribozymes cut the RNA transcript, separating the FLUC-expressing and the hRLUC-expressing RNA fragments from the sequence. An internal ribosome entry sequence (IRES) enhances translation of the uncapped hRLUC expression fragment, by replacing the functions of the 5′ cap and untranslated region (5′ UTR). An IRES sequence is optional, but preferred. The EMCV IRES we used in the prototype demonstrations conferred about a 10- to 50-fold increase in translation of an uncapped message, depending upon the particular reporter. For a very sensitive reporter such as a luciferase, the IRES may not be needed. Other IRES sequences are known in the art and may be used in practicing the invention, such as those from other picorna viruses or the Flaviviridae. The relative ratio of the reporter molecules is a measure of the impediment to transcription presented by a particular insert. If the RNA polymerase terminates transcription within an inserted test sequence, then the 3′ reporter will not be transcribed. The 5′ reporter (FLUC) is firefly luciferase. The 3′ reporter (hRLUC) is a humanized sea pansy luciferase. (Alternatively, any pair of reporters that can readily be quantified and distinguished from one another within the same cell or cell extract may be used. Many examples of reporter genes are known in the art.)

Expression of the 1.7 kb FLUC coding region in the first part of the transcription unit shows that the transcribing polymerase is well past “promoter escape” and into “processive elongation” before it encounters the test sequence. The inducible promoter and the tandem vector design are such that the expression of the control reporter and the test reporter result from transcription by the same polymerase, so that the ratio of expression of the two reporter molecules reflects the extent of termination as mediated by the center (“linker”) fragment. It is preferred that self-cleaving ribozyme sequences should flank the test sequence, so that the transcribed termination sequence does not itself become part of either of the reporter mRNA fragments. Thereby, the sequences of both reporter mRNA fragments are cleaved from the test sequence that lies between the ribozymes. See FIG. 1A, beneath the large arrows.

In the prototype, the first reporter mRNA fragment contained firefly luciferase (FLUC), and had a 5′ cap, but lacked a polyadenylation signal, and hence lacked a poly(A) tail. An A₃₂ tract just 5′ of the self-cleaving ribozyme aided in translation of the 5′ FLUC expression cassette. FLUC functions as the control reporter and is expressed independently of the inserted sequence. The downstream hRLUC reporter lacked a 5′ cap, but its translation was aided by an internal ribosome entry site (IRES). Because a chromosomal location probably more closely replicates the environment of a native gene than would a location in an episomal location or transient transfection, we adopted the Invitrogen Flp-In™ T-REx system. The constructs were introduced into a chromosome by the site-specific Flp recombinase into a single genomic location to make stable cell lines. The chromosomal location and orientation was consistent for all tested inserts. The consistency of the location and orientation removed a potentially significant complicating factor from the interpretation of the experimental results.

Constitutive expression of the Tet-repressor in the T-REx HEK 293 cell line repressed the construct's promoter in the absence of tetracycline. To test the ability of a single integrated copy of the tandem construct to inducibly express both of the reporter genes simultaneously and reproducibly, extracts representing 15,000 cells from three independently isolated clonal lines bearing tandem reporters were assayed for luciferase activity.

FIG. 1B depicts the observed induction of FLUC and hRLUC activities in clonal cell lines. The bars depict mean luciferase activity per cell in Relative Light Units for FLUC and hRLUC from clonal cell lines that contain a single integrated copy of a control tandem reporter construct. Cells were cultured without (−) or with (+) added doxycycline for 24 hours to induce transcription from the promoter. Extracts from 15,000 cells were assayed for luciferase activity. The mean induction observed for FLUC expression was 237±29 fold, and 56±3 for hRLUC. The error bars indicate the S.E.M. for a sample number of three.

Extracts were prepared from cells that had been maintained in normal growth media (−) or treated with 1 microgram/mL doxycycline (+), and then harvested 24 hours after induction. Cell lines with a single integrated tandem construct showed strong expression of both FLUC and hRLUC reporters upon induction. Mean induction was 237±29 fold for FLUC expression, and 56±3 fold for hRLUC. The lower hRLUC induction level reflected a higher background for hRLUC expression due to the nature of the hRLUC cassette, which is essentially a transcription trap. Whereas the FLUC RNA fragment has cap-dependent translation, and must initiate at or near the inducible start site to be translated efficiently, the combination of the ribozyme and the IRES allow translation of the hRLUC fragment even if transcription starts far upstream of the inducible start site. This background level is not generally a problem, because hRLUC expression was still induced over fifty-fold upon de-repression of the promoter. Thus, when the inducer is present, at least 98% of the transcribing polymerases initiated at the promoter, traversed the FLUC sequence, and traversed the intervening sequences before transcribing the hRLUC sequence. The mean induced FLUC and hRLUC activities were similar between individual cell lines (FIG. 1B), and we suspect that much of the variability that we did observe was artifactual, resulting from variability in counting and plating cells. More importantly, the ratio of hRLUC to FLUC luminescence was highly reproducible from one clone to another. This ratio was calculated for each well from sequential reads of the two luciferase values, and was independent of cell number. For example, clonal cell lines 1, 2 and 3 had hRLUC/FLUC ratios of 6.19, 6.27 and 6.28, respectively, which were not significantly different (p>0.05).

The self-cleaving hammerhead ribozyme used in these prototype constructs cleaves itself efficiently during in vitro transcription reactions, and presumably does so in vivo as well. See Grabczyk E, Usdin K (2000). The GAA•TTC triplet repeat expanded in Friedreich's ataxia impedes transcription elongation by T7 RNA polymerase in a length and supercoil dependent manner. Nucleic Acids Res 28: 2815-2822; Grabczyk E, Mancuso M, Sammarco M C (2007). A persistent RNA.DNA hybrid formed by transcription of the Friedreich ataxia triplet repeat in live bacteria, and by T7 RNAP in vitro. Nucleic Acids Res 35: 5351-5359; and Ditlevson J V, Tornaletti S, Belotserkovskii B P, Teijeiro V, Wang G, et al. (2008). Inhibitory effect of a short Z-DNA forming sequence on transcription elongation by T7 RNA polymerase. Nucleic Acids Res 36: 3163-3170.

We also confirmed ribozyme cleavage in vivo. PCR amplification of reverse-transcribed RNA prepared from cells harboring constructs with or without ribozymes showed a distinct difference between the two conditions. Primers designed to span the ribozymes yielded little or no PCR product, demonstrating that the mRNA produced by the product was successfully cleaved by the ribozymes. By contrast, PCR amplification with the same primers on a similar construct lacking the ribozymes (TAN NR) yielded a substantial PCR product. Our observations indicated that the ribozymes in the tandem constructs cleaved efficiently, and that luciferase reporters were expressed from the fragmented mRNA in sufficient quantity for use in a 96-well or other high-throughput format.

Poly(A)-Associated Termination is Directly Correlated with Downstream Flanking DNA

To test the effectiveness of the novel system in measuring transcription termination, we chose two well-characterized polyadenylation/termination regions of human origin. The relationship between transcription termination and polyadenylation is well established in both the human beta-globin (HBB) gene and the human beta-actin (ACTB) gene. We used the novel construct to quantify transcription termination in defined sections of polyadenylation sequences from both HBB and ACTB.

FIG. 2A depicts schematically the DNA inserts used with the prototype tandem vectors, indicating relative sizes. Fragments of green fluorescent protein, red fluorescent protein, and chloramphenicol acetyl transferase coding regions were not full-length, and did not make functional proteins. The polyadenylation regions used in these experiments are shown, approximately to scale. The position of the poly(A) addition site is indicated by a vertical black bar. The mini actin polyadenylation insert (MnipA) is about the width of that bar.

As a control, we used a tandem vector containing partial coding regions of red fluorescent protein (dsred). This widely used reporter gene's coding region was thought unlikely to contain functioning termination elements, and was further thought unlikely to contain functioning promoter elements that might contribute to background transcription. We compared the empty TAN0 control construct to tandem constructs containing either a single, double or tetrameric dsred fragment, and found no difference in transcription elongation (data not shown). We used the longest template in the dsred series as the control, as it exhibited little termination and yet was over a kilobase longer than the longest test templates (FIG. 2A, top).

All ACTB constructs showed robust transcription termination in the novel system. The TAN ACT1 construct contained only 400 bp of the ACTB poly(A) region, and yet terminated over 98% of transcribing polymerases, as measured by the change in the hRLUC/FLUC expression ratio as compared to the tandem construct with the dsred tetramer control insert.

FIG. 2B depicts the ratios of expressed hRLUC activity to FLUC activity for the indicated constructs in stable cell lines. The inclusion of approximately 400 to 2000 bp of sequence containing either the actin (ACT1-3) or globin (HBB1-3) poly(A) addition site effectively terminated transcription elongation. Transcription was induced with doxycycline, and cells were harvested after 24 hours. Results for each cell line are expressed as a percentage of the dsred control cell line, which did not contain a polyadenylation sequence. Longer poly(A) region fragments that included a putative co-transcription cleavage element (CoTC) were included in the globin constructs, and reduced read-through expression of the downstream reporter to ˜1-2% of the control dsred cell line for all constructs. A minimal poly(A) signal sequence provided modest transcription termination (MnipA). The control (MnopA) contained a nearly identical sequence, except for two changed bases in the hexamer portion of the polyadenylation signal. The y-axis values are hRLUC/FLUC expression ratios, normalized versus a positive cell lysate run for each plate. All of the changes were significant (p<0.05) as compared to the dsred control. Error bars indicate the S.E.M. for a sample number of three.

The ACTB constructs showed greater transcription termination when more sequence downstream of the poly(A) addition site was included in the construct. ACT2 was approximately 3-fold more effective than ACT1, and ACT3 was about 4-fold more effective. See FIG. 2B.

Analysis of the human HBB poly(A) region showed a similar trend, with increased termination correlating with increased sequence length (FIG. 2B). When the ACT1 sequence was compared to the HBB1 sequence, there was nearly a ten-fold difference in transcription read-through, indicating that the ACTB sequence terminated elongation more effectively within a shorter sequence length. However, HBB2 has an additional 1100 base pairs of HBB gene sequence, including a putative co-transcriptional cleavage (CoTC) site. HBB2 terminates transcription about 36 times more effectively than HBB1. Termination by HBB2 is at least as effective as that of the like-sized ACT2. However, further addition of downstream sequences in HBB3 did not greatly increase the degree of termination. This observation suggested that either we had included the majority of termination signals within a little over one kilobase downstream of the poly(A) site, or that we had reached the limit of detection in this embodiment of the novel system. Similarly, we saw no significant difference in transcription termination between the strongest terminators we tested in the tandem vectors, ACT3 and HBB3. Because we have no reason to believe that the longest termination sequences we tested from two different genes should provide precisely the same degree of termination, we concluded that 99.6% termination lay close to the limit of detection in this particular embodiment of the novel system.

Because the shortest ACTB sequence that we have tested, TAN ACT1, was very effective at terminating transcription elongation within just a few hundred base pairs, we also decided to test a minimal polyadenylation signal (MnipA) that was derived from the core of ACTB. This 59 base-pair sequence contained the AATAAA hexamer signal, the poly(A) addition site, and the most proximal downstream GT-rich tract. The MnipA sequence showed a 25% decrease from the TAN dsred control construct. See FIG. 2B. An alternative sequence, MnopA, was identical to MnipA except for two base pairs in the hexamer sequence (AATATT instead of AATAAA). Surprisingly, MnopA retained some ability to terminate, showing a modest decrease (12%) relative to control. See FIG. 2B. Given the statistical probability of occurrence of AATAAA hexamers and GT-rich tracts in a genome, the modest termination afforded by the minimal poly(A) region itself is not implausible, and supports the hypothesis that multiple signals are needed for strong termination to prevent small mutations from truncating transcription units.

The Role of Xrn2 and Senataxin in Transcription Termination

The self-cleaving ribozymes of the novel construct not only leave a 5′ OH following cleavage, but they also have a structured end. Assuming that Xrn2 shows the same preference as its yeast homologue Rat1p, then the novel ribozyme-containing construct should allow one to short circuit Xrn2 activity. If ribozyme cleavage precedes the arrival of Xrn2, then the transcribing polymerase would “escape” the “chase-down,” and hRLUC would be expressed. It was possible that part of the length-dependence effect seen in FIG. 2B may have been due to ribozyme cleavage short-circuiting the “torpedo” model. To determine whether this was indeed the case, we added spacer DNA between the ribozymes and the poly(A) signals by cloning the poly(A) regions within a stretch of polylinker remaining in the TAN dsred control vector. See FIG. 2A, top.

Surprisingly, the presence of dsred sequence 3′ to the poly(A) regions decreased the effectiveness of transcription termination in all constructs tested.

FIG. 3A depicts hRLUC/FLUC expression ratios with the constructs indicated. The inclusion of 2.6 kilobases of dsred tetrameric sequence downstream (3′) from the ACT and HBB sequences provided modest release from transcription termination. Transcription was induced with doxycycline, and cells harvested after 24 hours. The y-axis values are hRLUC/FLUC expression ratios normalized versus a positive cell lysate run for each plate, and expressed as a percentage of the dsred control cell line. All of the changes were significant (p<0.05) as compared to the dsred control. The error bars indicate the S.E.M. for a sample number of three.

The effect of the additional downstream sequence was greater on ACT1, reducing termination 5-fold while reducing termination of HBB1 only 2-fold. Compare FIGS. 2B and 3A. This trend continued, with the addition of dsred spacer reducing termination in the ACT2 and ACT3 constructs approximately 3-fold; while the strongest terminator of the HBB series, HBB3, was affected less than 2-fold. In the presence of the downstream dsred spacer, HBB2 and HBB3 (both of which include the CoTC) are more effective than ACT2 and ACT3 respectively (p<0.05). See FIG. 3A. This differential response to the addition of downstream dsred sequences suggests that the effect of the dsred spacer cannot simply be attributed to adventitious promoters within the spacer. If transcription initiated within the dsred spacers to increase hRLUC expression, then the effect would be expected to be highest in those constructs with the most repressed hRLUC, such as HBB3, and lowest in the constructs with a fair amount of read-through to hRLUC, such as ACT1 and HBB1.

To confirm that the luciferase reporter levels were representative of FLUC and hRLUC mRNA levels, and were not merely a reflection of changes in overall translation attributable to the added dsred sequence, we used real-time PCR to quantify luciferase mRNA.

The constructs caused changes in FLUC and hRLUC mRNA levels. FIG. 3B depicts measurements from real-time RT-PCR analysis. FLUC and hRLUC mRNA were measured by real-time RT-PCR, expressed as the relative ratio of hRLUC/FLUC mRNA; in both cases normalized as a percentage of levels for the dsred control cell line. This ratio was observed to decrease as larger segments of polyadenylation sequence were included in the tandem construct. Error bars indicate the S.E.M. for a sample number of three. The data showed that our novel constructs allow one to quantify transcription elongation and termination.

Transcription termination with the novel constructs was generally less effective with the addition of downstream dsred sequence, counter to what would be expected if the Xrn2 “torpedo” model were a major contributor to termination. To better determine the degree to which Xrn2 contributed to termination in our system, we used short hairpin RNA (shRNA) to knock down Xrn2, and evaluated the effects in the cell lines. Knockdowns in the cell lines were performed using a commercially-available expression plasmid (pLKO), and insert sequences developed by the RNAi Consortium (TRC) and chosen from the NCBI probe website. We performed Western blots on protein extracts from cells treated either with the empty vector pLKO.1, or with the shRNA-bearing vector to confirm shRNA-specific knockdown of Xrn2. Xrn2 was knocked down to similarly low levels in cell lines ACT1, ACT3, ACT1/dsred, ACT3/dsred, HBB1, HBB3, HBB1/dsred, and HBB3/dsred.

FIG. 4 depicts hRLUC/FLUC expression ratios with the constructs indicated. Neither Xrn2 nor senataxin knockdown provided much release from transcription termination. The y-axis values are hRLUC/FLUC expression ratios normalized versus a positive cell lysate run for each plate. Error bars indicate the S.E.M. for a sample number of three. Significant differences (p<0.05) from the corresponding vector (pLKO)-treated samples are indicated by asterisks.

Knockdown of Xrn2 in ACT1, ACT3, ACT3/dsred, and HBB3/dsred showed no statistically significant difference from control with regard to transcription elongation (p>0.05). Act1/dsred, HBB1, HBB1/dsred, and HBB3 showed a minimal increase in transcription when Xrn2 was knocked down in the cell line (p<0.05).

While decreased Xrn2 did affect transcription elongation in some of the constructs, it provided only a modest increase in read-through, which indicated that a role is played by other proteins in transcription termination. Senataxin (Sen1) has considerable homology to Sen1p, which is a helicase that is essential for processing RNA in yeast, and that has been implicated in transcription regulation. To test whether senataxin contributes to transcription termination in human cells, we knocked down senataxin levels in the same cell lines to evaluate the resulting effect on transcription elongation. See FIG. 4B.

Senataxin levels were knocked down using pLKO and TRC shRNA sequences. Readily available antibodies to human senataxin did not prove to be reliable in Western blots, so instead we confirmed knockdown of senataxin using real-time PCR. Quantitative PCR indicated that senataxin levels following shRNA treatment were less than 20% of the levels in pLKO.1-treated cells (16±3%). Knockdown of senataxin in the cell lines increased transcription elongation, in the absence of the dsred spacer sequence, for the poly(A)-containing constructs ACT1, HBB1, and HBB3, but not for ACT3. See FIG. 4. When a dsred spacer sequence was included, all four constructs showed a significant (p<0.05) albeit small increase in transcription elongation.

Summary of Prototype Experiments

Our prototype experiments showed that the novel system was capable of probing template-mediated influences on the dynamic balance between elongation and termination by RNAP II. We have measured the termination associated with two well-characterized polyadenylation signals. We compared defined regions surrounding the human beta-actin and beta-globin polyadenylation sites, and confirmed that even short segments were effective at terminating transcription. Using shRNA we knocked down two candidate proteins involved in transcription termination, but in each case observed only modest effects. While we had expected that adding a length of heterologous sequence downstream of the polyadenylation signals would increase termination, our observations showed that the reverse was actually true. In addition, the magnitude of the response to the downstream sequence was gene dependent, with the ACTB sequences showing a greater release from termination.

### BRIEF DESCRIPTION OF THE DRAWINGS

FIG. 1A depicts schematically a prototype embodiment of the invention.

FIG. 1B depicts the observed induction of FLUC and hRLUC activities in clonal cell lines.

FIG. 2A depicts schematically the DNA inserts used with the prototype tandem vectors, indicating relative sizes.

FIG. 2B depicts the ratios of expressed hRLUC activity to FLUC activity for the indicated constructs in stable cell lines.

FIG. 3A depicts hRLUC/FLUC expression ratios with the constructs indicated.

FIG. 3B depicts measurements of FLUC and hRLUC mRNA from real-time RT-PCR analysis.

FIG. 4 depicts hRLUC/FLUC expression ratios with the constructs indicated.

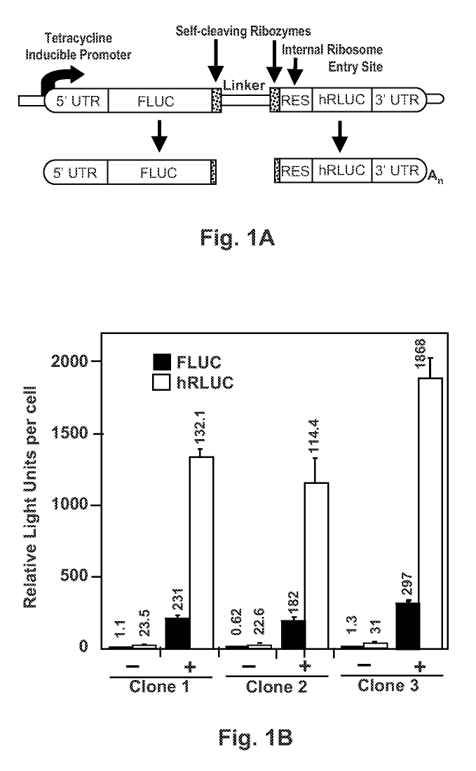

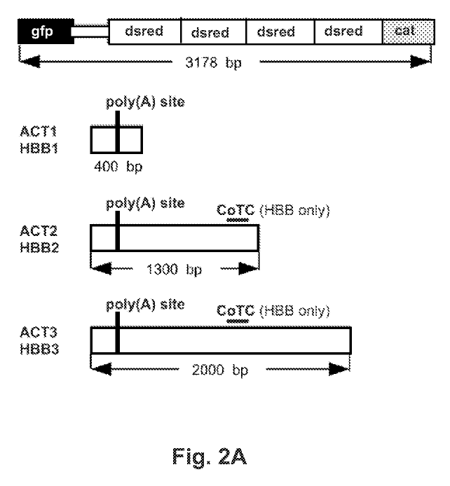

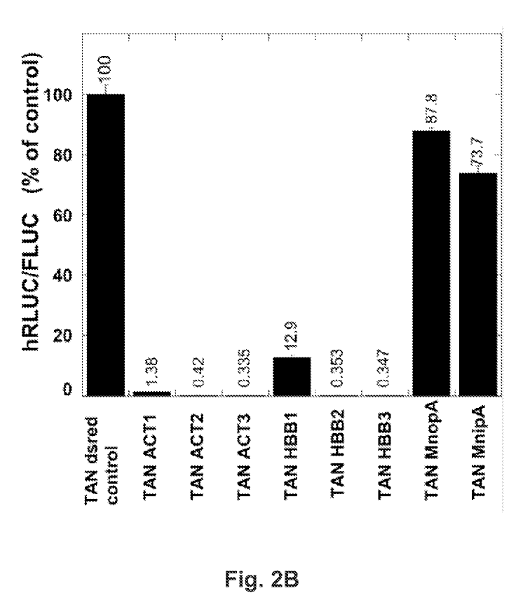

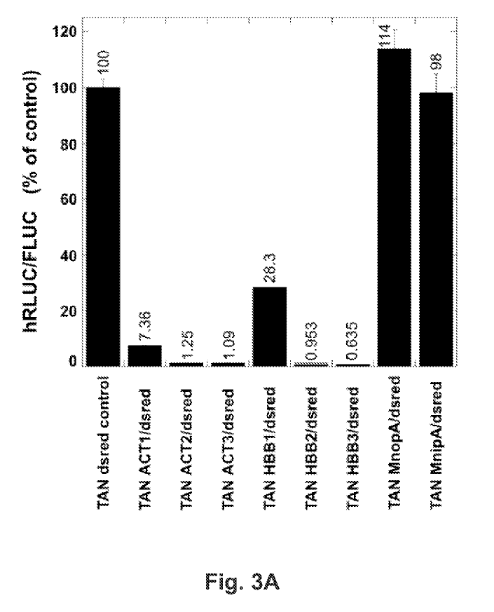

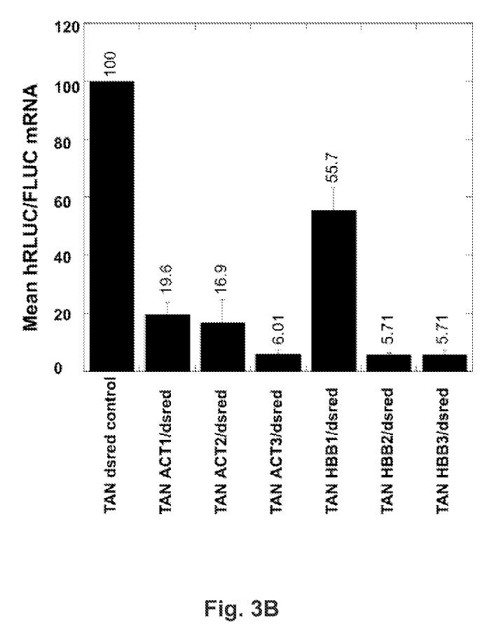

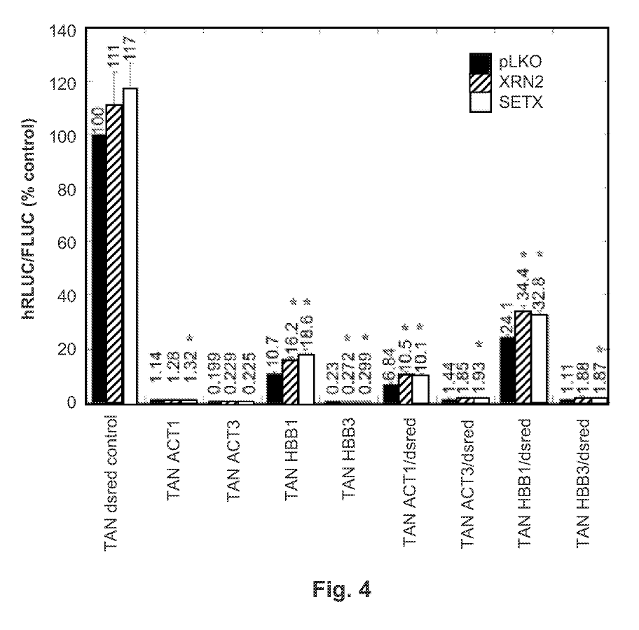

### MODES FOR CARRYING OUT THE INVENTION

### Example 1

### Materials

Restriction enzymes were purchased from New England Biolabs (Ipswich, Mass.). All tissue culture reagents were purchased from Invitrogen (Carlsbad, Calif.) unless otherwise stated. All other chemicals were purchased from Sigma unless otherwise stated.

Construction of Plasmids/Vectors

### Examples 2-6

We constructed a tandem reporter construct in pcDNA5/FRT/TO (Invitrogen, Carlsbad, Calif.). The CMVIE promoter in pcDNA5/FRT/TO contained two Tet-operator sequences at the start of transcription, working in conjunction with the constitutively-expressed tetracycline repressor in the T-REx cell lines (Invitrogen). To insert the reporters downstream of the pcDNA5/FRT/TO promoter the plasmid was digested with HindIII & BamH1; and a HindIII & BamH1 fragment containing firefly luciferase (FLUC) from pGL3-control (Promega, Madison, Wis.) was inserted to make pcDN/FRT/FL.

A 160 bp self-cleaving ribozyme sequence was PCR amplified with primers that add SpeI & EcoRI sites 5′ and MfeI, BglII & XbaI sites 3′. The 160 bp sequence was as described in Grabczyk E, Usdin K (2000) The GAA•TTC triplet repeat expanded in Friedreich's ataxia impedes transcription elongation by T7 RNA polymerase in a length and supercoil dependent manner. Nucleic Acids Res 28: 2815-2822; and Grabczyk E, Mancuso M, Sammarco M C (2007) A persistent RNA.DNA hybrid formed by transcription of the Friedreich ataxia triplet repeat in live bacteria, and by T7 RNAP in vitro. Nucleic Acids Res 35: 5351-5359.

Ribozyme PCR product cut with SpeI & BglII was ligated into XbaI & BamH1 digested pcDN/FRT/FL to create pcDN/FRT/FL/RZ. EcoRI- & BglII-cut ribozyme PCR product was inserted just 5′ of the IRES in pIRES2-EGFP (Clontech, Palo Alto, Calif., after digestion with EcoRI & BamH1). The resulting 5′/ribozyme/IRES-3′ fragment was excised using NheI & NcoI (at the ATG site of IRES), and ligated into NheI- & NcoI-partially digested phRL-TK (Promega), a plasmid that contains humanized renilla luciferase. An XhoI- & XbaI-digested 1720 bp fragment with this ribozyme/IRES/hRLUC sequence was then ligated into pcDN/FRT/FL/RZ, and digested with XhoI & XbaI to yield the pcDN/FRT/FL/RZ/RZ/IRES/hRL (or TAN0) plasmid. Unique NotI and XhoI sites remaining between the ribozymes were expanded with oligonucleotides into a polylinker (5′-NotI, NheI, BamH1, XmaI, XhoI-3′) to make TAN1. A control tandem vector with no ribozymes was made by cutting TAN0 with MfeI (partial) and EcoR1 (partial), dropping out both ribozymes, removing the polylinker site, and creating compatible sticky ends.

Coding sequence spacers: PCR was used to add XbaI to one side of a fragment of the green fluorescent protein (GFP) coding sequence from pIRES2-EGFP (1695-1254 bp on the plasmid from Clontech); and NotI, NheI, and BamH1 sites were added to the other side. This PCR product was cut with XbaI & BamH1 and inserted into pREX that had been cut with BglII & SpeI. The plasmid pREX is described in Grabczyk E, Usdin K (1999). Generation of microgram quantities of trinucleotide repeat tracts of defined length, interspersion pattern, and orientation. Analytical Biochemistry 267: 241-243.

PCR was used to add BamH1, XmaI and XhoI sites to one side of a 361 bp fragment of the chloramphenicol acetyltransferase (CAT) coding sequence from pSV2CAT (4969-4608 bp Genbank M77788), and a XbaI site to the other side. This CAT fragment PCR product and the plasmid with the GFP fragment were cut with BamH1 & XbaI and joined to make pREX-GC. PCR was used to generate a 529 bp fragment of the dsred coding region from pDsRed1-Mito (700-1229 on the plasmid from Clontech), adding BamH1 and XbaI sites to one side, and a NheI site to the other. PCR fragments cut with XbaI, NheI, BamH1, and NheI were mixed, ligated, cut again with BamH1 and NheI, and gel-purified. A 2158 bp dsred tetramer was ligated into a GFP-CAT fragment construct digested with Nhe1 and BamH1. The dsred tetramer insert (with flanking GFP and CAT fragments) was PCR amplified and inserted into the polylinker region of the Tan1 construct. The non-human sequences (GFP/CAT) flanking the polylinker site serve as unique priming sites. The tetramer fragment serves as a size control spacer sequence.

### Examples 7-11

The polyadenylation regions of the human beta-globin (HBB, NM_{—}000518) and beta-actin (ACTB, NM_{—}001101) genes were isolated via PCR. A first PCR round used primers generated by Primer3 to amplify sequences from genomic DNA samples. In the numbering scheme used here, the poly(A) addition site for each gene is numbered +1, which corresponds to base 5203272 on human chromosome 11 for HBB, and to base 5533305 on human chromosome 7 for ACTB, using the March 2006 numbering system. Primer sets were paired as follows, HBB-513F with HBB+2390R, and ACT-449F with ACT+1826R. The second PCR round used product from the first round, and employed new primer sets that generated restriction enzyme sites (Nhe I and Not I) at the very ends of the product for later use for cloning. HBB-200NotF primer was paired with each of the following: HBB+200NheR, HBB+1100NheR, and HBB+1800NheR to make products of 400, 1300, and 2000 base pairs, respectively, named HBB1, HBB2, and HBB3. The ACT-200NotF primer was paired with ACT+200NheR, ACT+1100NheR and ACT+1800NheR (reverse) primers to generate products 400, 1300, and 2000 bp long, called ACT1, ACT2 and ACT3 respectively.

A minimal poly(A) addition site (MniACT) taken from the core of the ACTB site was made by annealing the oligodeoxyribonucleotides MniACTpA1 and MniACTpA2. Versions with the polyadenylation signal mutated were made with the oligodeoxyribonucleotides Mni0ACTpA1 and Mni0ACTpA2. The sequences for the oligodeoxyribonucleotides MniACTpA1, MniACTpA2, Mni0ACTpA1, and Mni0ACTpA2 may be found in U.S. priority application 61/361,710 at page 19, hereby incorporated by reference.

Cell Transfections and Establishment of Stable Cell Lines

### Examples 12-13

To integrate the constructs into a stable cell line in a consistent chromosomal location, we used the Invitrogen Flp-In T-REx system as described in Sammarco M C, Grabczyk E (2005). A series of bidirectional tetracycline-inducible promoters provides coordinated protein expression. Anal Biochem 346: 210-216. All cell lines were maintained in Dulbecco's modified Eagle's medium (DMEM) with high glucose and 10% FBS (Hyclone, Logan, Utah). Cell lines were induced with 1 μg/ml doxycycline (Sigma) for 24 hours before use in the luciferase experiments.

shRNA Knockdown of SETX and XRN2

### Examples 14-15

Cells were plated in a 24-well plate and transfected with 250 ng of shRNA construct using lipofectamine 2000 (Invitrogen) as per the manufacturer's protocol. The shRNA constructs cloned into the pLKO.1 vector were from Open Biosystems:

XRN2 shRNA: TRCN0000049899 (NM_{—}012255 mRNA)
SETX shRNA: TRCN0000051517 (NM_{—}015046 mRNA)

Transfected cells were split into a 100 mm tissue culture dish 24 hours post-transfection, and selected with 1 μg/ml of puromycin for 5 days. Knockdowns were confirmed using Western blots and qRT-PCR for XRN2 and SETX respectively.

Western Blot Analysis of XRN2

### Example 16

Cells were scraped and lysed in 2X Laemmli Buffer (20% glycerol, 2% SDS, 100 mM Tris (pH 6.8), fresh 125 mM DTT). XRN2 Western blots were performed by resolving 150 μg of protein on an 8% SDS-PAGE gel (37.5:1) (Bio-Rad Mini Protean™ system). The samples were transferred to Immobilon-P membrane (Millipore) using a Bio-Rad semi-dry transfer apparatus. Membranes were blocked for an hour at room temperature in 20% evaporated Carnation Milk/PBS mixture, followed by overnight incubation at 4° C. with the primary antibodies.

Rabbit anti-human XRN2 primary antibodies (Bethyl labs), and mouse anti-β-actin primary antibodies (Sigma) were used at 1:250 and 1:5000 dilutions, respectively. Goat anti-rabbit, horseradish peroxidase-conjugated secondary antibody (Pierce), and goat anti-mouse, horseradish peroxidase-conjugated secondary antibody (Molecular probes) were each used at a dilution of 1:10000, followed by visualization using ECL Advance™ (Amersham). Images were obtained with the Kodak Gel Logic 440 Imaging system, and were analyzed with Kodak Molecular Imaging software (Version 4.0).

Dual Luciferase Assay

### Example 17

The dual luciferase reagent (DLR) kit from Promega was used according to the manufacturer's directions, as described in greater detail in Sammarco M C, Grabczyk E (2005). A series of bidirectional tetracycline-inducible promoters provides coordinated protein expression. Anal Biochem 346: 210-216.

Real Time Reverse Transcription-PCR

### Examples 18-21

Synthesis of cDNA to quantify FLUC and hRLUC expression in the TAN and

TAN/dsred cell lines was performed directly from cell lysates using the SuperScript™ III Cells Direct cDNA Synthesis System (Invitrogen). To quantify senataxin knockdown, RNA was obtained with TRI-Reagent (Molecular Research Center, Inc.). First strand cDNA synthesis was done with 5 ng of RNA template using the Sidestep™ II QPCR cDNA synthesis kit (Stratagene).

The primers, FLUC sense, FLUC antisense, hRLUC sense, hRLUC antisense, SETX sense, SETX antisense, 18S rRNA sense, and 18S rRNA antisense, were purchased from IDT (Coralville, Iowa) and used at final concentrations of 400 nM. The sequences for these primers may be found in U.S. priority application 61/361,710 at page 21, hereby incorporated by reference. The iQ SYBR Green Supermix was used according to the manufacturer's recommendations (Biorad, Hercules, Calif.). PCR conditions were an initial 10 min denaturing at 95° C., and then 40 cycles of: 95° C. for 30 sec, 55° C. for 1 min, and 72° C. for 30 sec. Standard curves for the FLUC and hRLUC primer set were made with dilutions of the Tandem dsred tetramer control plasmid as described in Sammarco M C, Ditch S, Banerjee A, Grabczyk E (2008) Ferritin L and H Subunits Are Differentially Regulated on a Post-transcriptional Level. J Biol Chem 283: 4578-4587. Senataxin knockdowns were confirmed by the ΔΔCt method using 18S rRNA as an internal control, as described in Livak K J, Schmittgen T D (2001). Analysis of relative gene expression data using real-time quantitative PCR and the 2(-Delta Delta C(T)) Method. Methods 25: 402-408; and Winer J, Jung C K, Shackel I, Williams P M (1999). Development and validation of real-time quantitative reverse transcriptase-polymerase chain reaction for monitoring gene expression in cardiac myocytes in vitro. Anal Biochem 270: 41-49. Data were analyzed using the Mx3000P software (version 2.0).

Autosomal Dominant Polycystic Kidney Disease

One example of the clinical utility of the invention is in the diagnosis and treatment of Autosomal Dominant Polycystic Kidney Disease. ADPKD is one of the most common genetic diseases, affecting one in every 500 people of all ethnic groups worldwide. Mutations in the PKD1 gene cause 85% of ADPKD. Despite the name, ADPKD is a recessive disease in which one mutant allele is inherited and the second allele acquires a mutation. The appearance of dominant inheritance is due to a very high rate of somatic mutation in the second allele. The reason for the high rate of PKD1 somatic mutation is unclear. There is an urgent need to understand and reduce this rate. Reduced PKD1 mutation will directly translate to reduced cyst formation.

We hypothesize that antisense transcription within regions of the PKD1 gene directly contributes to mutation of the PKD1 gene. ADPKD affects over 600,000 U.S. citizens, and far more worldwide. PKD1 patients progress to end-stage renal disease at an average age of 54, requiring chronic or extreme interventions such as dialysis and kidney transplants. The cost of PKD in human suffering and shortened productive lives is massive. This invention may be used in the identification of prognostic markers for cyst formation and new therapeutic targets to prevent mutational loss of PKD1 gene function, and to reduce the burden of this disease.

The “two-hit” model of ADPKD involving somatic mutation of PKD1 has a great deal of support. Frequent loss of heterozygosity (LOH) at PKD1 also suggests enhanced gene conversion at or near this locus. Furthermore, at least six segmental duplications of PKD1 are known from exons 1 to 33, an observation that is consistent with the existence of a source for gene conversion events. Repetitive regions within PKD1 may have triplex-forming potential, but it is not clear whether transitions to unusual DNA structures such as these may be linked to a mutational mechanism for PKD1. Unfortunately, there has been little recent research in the area of DNA structures formed by the PKD1 repeats, leading perhaps to the impression that the field has already been fully explored and has become a dead end.

Our hypothesis is that an RNA•DNA hybrid is formed by antisense transcription within regions of the PKD1 gene, and that that the RNA•DNA hybrid directly contributes to mutation of the PKD1 gene. We will demonstrate the causal link between DNA repeats and somatic mutation in the PKD1 gene. By analogy, gene conversion and somatic hyper-mutation play a role in the generation of antibody diversity. These processes are transcription-dependent. Transcription is thought to make the DNA more accessible to modifying enzymes. Our novel mutational model explains the frequent loss of heterozygosity in PKD1 via enhanced gene conversion into a mutant allele. Our new model also accounts for the frequent existence of novel PKD1 mutations, due to enhanced recombination with the six PKD1-like sequences elsewhere on chromosome 16. Finally, novel PKD1 mutations can be introduced at the RNA•DNA hybrid directly by cytosine deamination by an AID-like activity in the kidney. This novel approach may also be used in assessing mutation potential in other critical genes throughout the human genome or other genome.

Our hypothesis is that antisense transcription in the PKD1 gene causes the formation of recombinogenic and mutagenic structures in the DNA template, somewhat analogous to what occurs in immunoglobulin genes. There are repetitive G•C rich regions throughout PKD1. These G•C rich regions are oriented so that normal PKD1 transcription makes C-rich transcripts, which typically do not tend to form anomalous secondary structures. However, if these regions are transcribed in the antisense direction, they can resemble the G-rich regions of immunoglobulin switch regions. Gene conversion and somatic hyper-mutation play a role in the generation of antibody diversity. These processes are transcription-dependent. Transcription within switch regions creates structures such as RNA•DNA hybrids (R-loops) that targets the DNA for subsequent modification or recombination. For example, the neighboring tuberous sclerosis complex gene (TSC2) is one potential source of antisense PKD1 transcription. TSC2 and PKD1 are transcribed toward one other, and they have 3′ ends that are only 63 base pairs apart.

### Examples 22-23

To demonstrate this hypothesis, we shall demonstrate two specific points:
1. We will confirm the degree to which antisense transcription promotes secondary structures in PKD1 transcripts. Repeats from PKD1 introns 1, 21, and 42 will be evaluated for structure formation resulting from in vitro transcription in either direction, by a combination of gel mobility studies, susceptibility to enzymatic and chemical modification, and electron microscopy.
2. We will confirm the extent to which leaky transcription termination at the end of TSC2 acts as a source for PKD1 antisense transcription. Functional characterization of transcription read-through from TSC2 polyadenylation regions into PKD1 will be performed using DNA derived from PKD and control cells cloned into the novel tandem reporter construct to measure transcription read-through. The constructs will be transfected into both PKD and normal cell lines to identify cis- and trans-modifiers of TSC2 transcription termination.

Because of the pronounced G•C strand asymmetry of the repeats within PKD1, we expect to see strong, orientation-dependent formation of structures respecting item 1. Transcription can continue on for thousands of bases, sometimes even through very strong terminators; our prediction is that we will see substantial evidence of antisense transcription from the 3′ end of PKD1 in point 2. Confirmation of these two points will provide a major paradigm shift in current thinking about models of ADPKD causation.

### Example 24

This work will identify prognostic markers for cyst formation and new therapeutic targets to prevent mutational loss of PKD1 gene function and thereby reduce cyst formation. ADPKD patients inherit one mutated copy of PKD1; and a cyst appears when the second copy also acquires a mutation. The high mutability of the human PKD1 gene suggests that an active process is involved. Strand-asymmetric DNA repeats located throughout the human PKD1 locus may contribute to its exceptionally high mutability, by providing initiation points for RNA•DNA hybrids. Antisense transcription due to inefficient termination of TSC2 transcription may be the active agent initiating the mutational process. The novel tandem assay system may be used to confirm whether sequence differences in the TSC2-PKD1 termination region are linked to inefficient TSC2 termination, and to provide therapeutic targets for pharmaceutical compounds to slow the progression of ADPKD or to prevent its onset in persons who carry one mutant allele. Cis-mutations that alter TSC2 transcription termination will have immediate prognostic value. For example, use of the novel tandem reporter system with ADPKD patient-derived sequences may indicate that one or more single-nucleotide polymorphisms (SNP) is linked to leaky termination of TSC2 transcription. Knowledge of such SNPs can allow one to predict the time-course and severity of a patient's likely ADPKD. Knowledge of such SNPs, gained through the use of the tandem reporter system, may then be used to prepare a simple genetic test through means otherwise known in the art to predict ADPKD likelihood, speed of progression and severity, e.g., as described in Example 26 below.

This work will demonstrate the extent to which read-through transcriptions past a transcription terminator contribute to ADPKD. Even if such events are only responsible for a small percentage of ADPKD cases, drugs targeting the read-through would represent a much larger market than all cases of Friedreich ataxia, for instance. The tandem reporter system can be used in high throughput screens to identify drugs that alter transcription read-through, e.g., as in Example 27; or to identify protein targets that alter transcription read-through, e.g. as in Examples 29 and 30.

### Example 25

The tandem reporters are used, for example, in a functional assay to confirm the degree to which cis-acting factors or trans-acting factors alter the efficiency of transcription termination from the TSC2 gene, as outlined in point 2 above.

### Example 26

Where cis-acting factors (e.g., point mutations, deletions, or insertions in or near the 3′ end of the TSC2 transcription unit; or in the neighboring PKD1 sequence in DNA from ADPKD patient samples) are shown to affect the efficiency of transcription termination using the functional assay, the mutations are more specifically identified by sequencing via techniques known in the art. Diagnostic genetic tests for the specific mutations thus identified are then used to test at-risk populations for ADPKD quickly and inexpensively.

### Example 27

The tandem reporters are also used, for example, in a method for screening pharmaceutical compounds for activity against cis-acting mutations in the TSC2-PKD1 sequence, or other genomic sequence of interest. Cells carrying a single copy of the mutant TSC2-PKD1 sequence with the novel tandem vector integrated at a single genomic location are compared to control cells carrying the wild-type sequence integrated at the same genomic location. For example the HEK 293 derivatives described in Banerjee, A., Sammarco, M. C., Ditch, S., Wang, J., and Grabczyk, E. (2009). A Novel Tandem Reporter Quantifies RNA Polymerase II Termination in Mammalian Cells. PLoS ONE 4, e6193 might be used for this purpose.

### Example 28

We have tested a dual luciferase assay using the novel tandem vector in a 1536-well plate format using sequential application and reading of the dual-glo reagents to activate the two luciferase reporters. The assay gave good Z-prime values at 1000 to 2000 cells per well in 5 microliters of media. The HEK 293 cells can tolerate low serum levels (˜0.5%), if desired. The assay tolerates the commonly used solvent dimethyl sulfoxide (DMSO) over the 24 and 48-hour testing periods. The assay may be used in both 20 nanoliter and 50 nanoliter pin application of chemical libraries for 1536-well plate format high-throughput screens.

### Example 29

In a cell-based assay, micro RNA, siRNA or shRNA libraries may be screened, for example in high throughput 1536-well plate formats to identify proteins that alter transcription termination or read-through. The screen can examine both cis-acting and trans-acting effects.

### Example 30

Screening for trans-acting influences can also be performed directly in other cell lines, such as ADPKD-derived patient cells. The cells are preferably first immortalized through techniques well known in the art; and it is preferred first to confirm that the immortalized cells are well suited for use in high throughput conditions.

Areas of application for the novel system include, among others:
1) High throughput assays to identify transcription terminators.
2) High throughput assays to measure transcription terminator efficiency.
3) High throughput assays to identify drugs that increase the efficiency of a transcription terminator.
4) High throughput assays to identify drugs that decrease the efficiency of a transcription terminator.

The complete disclosures of all references cited in this specification are hereby incorporated by reference. Also incorporated by reference is the complete disclosure of the priority application, U.S. provisional patent application Ser. No. 61/361,710, filed 6 Jul. 2010. Also incorporated by reference is the following publication by the inventors and their co-workers: A. Banerjee, “A novel tandem reporter quantifies RNA polymerase II termination in mammalian cells,” PLoS One, vol. 4, no. 7, e6193 (9 Jul. 2009). In the event of an otherwise irreconcilable conflict, however, the present specification shall control.

What is claimed:

1. A construct comprising an isolated polydeoxyribonucleotide comprising, in the 5′-to-3′ direction in the following order: a transcriptional promoter, a region that encodes a first reporter molecule, a region that encodes a first self-cleaving ribozyme, a linker, a region that encodes a second self-cleaving ribozyme, and a region that encodes a second reporter molecule; wherein:
(a) when said polydeoxyribonucleotide, or a portion of said polydeoxyribonucleotide, is transcribed into RNA, then any portion of the resulting RNA molecule that includes a transcript of one of said self-cleaving ribozyme regions will be rapidly cleaved under physiological conditions; whereby the portion of the RNA molecule that includes the transcript for the first reporter molecule is rapidly cleaved from the portion of the RNA molecule, if any, that includes the transcript for the linker and the transcript for the second reporter molecule; and whereby the portion of the RNA molecule, if any, that includes the transcript for the second reporter molecule is rapidly cleaved from the portion of the RNA molecule that includes the transcript for the linker and the transcript for the first reporter molecule;
(b) the sequences of said first and second self-cleaving ribozyme regions may be the same or different;
(c) the encoded first and second reporter molecules are different, such that the first and second reporter molecules are readily distinguishable from one another;
(d) said linker comprises either a test polydeoxyribonucleotide whose effect upon RNA transcription termination or RNA transcription elongation is being assayed; or said linker comprises one or more restriction enzyme recognition sites into which such a test polydeoxyribonucleotide may be spliced; or both;
wherein said construct is adapted to be used to assay the effect of a test polydeoxyribonucleotide upon RNA transcription termination or RNA transcription elongation in an in vivo system or an ex vivo system, wherein the assay comprises the steps of:
(i) introducing the construct into the in vivo system or ex vivo system;
(ii) causing or allowing initiation of transcription of said construct into mRNA;
(iii) measuring the level of expression of the first reporter molecule;
(iv) measuring the level of expression of the second reporter molecule; and
(v) determining the ratio of the measured expression of the second reporter molecule to the measured expression of the first reporter molecule, as an assay of the level of RNA transcription termination or RNA transcription elongation in the in vivo system or ex vivo system, as modulated by the presence of the test polydeoxyribonucleotide, as compared to the same ratio measured for a control under similar conditions; wherein in the control the linker consists essentially of one or more restriction enzyme recognition sites, or wherein in the control construct the linker has no substantial effect on RNA transcription termination or RNA transcription elongation, and wherein the control is otherwise substantially similar to the construct.

2. The construct of claim 1, wherein said transcriptional promoter is an inducible promoter.

3. The construct of claim 1, wherein said region that encodes the first reporter molecule, said region that encodes the second reporter molecule, or both additionally comprise a polyadenylation region.

4. The construct of claim 1, wherein said region that encodes the second reporter molecule additionally comprises an internal ribosome entry sequence to enhance the translation of transcribed RNA that corresponds to the second reporter molecule sequence.

5. The construct of claim 1, wherein said region that encodes the first reporter molecule, said region that encodes the second reporter molecule, or both encode a luciferase.

6. A host cell that comprises the construct of claim 1.

7. The host cell of claim 6, wherein said construct is integrated into one of said host cell's chromosomes.

8. The host cell of claim 6, wherein said construct is integrated into a plasmid or other episome.

9. The host cell of claim 6, wherein said host cell is a mammalian cell.

10. A method of assaying the effect of a test polydeoxyribonucleotide upon RNA transcription termination or RNA transcription elongation in an in vivo system or an ex vivo system; wherein said assay comprises the steps of:
(i) introducing the construct of claim 1 into an in vivo system or ex vivo system;
(ii) causing or allowing initiation of transcription of the construct into mRNA;
(iii) measuring the level of expression of the first reporter molecule;
(iv) measuring the level of expression of the second reporter molecule; and
(v) determining the ratio of the measured expression of the second reporter molecule to the measured expression of the first reporter molecule, as an assay of the level of RNA transcription termination or RNA transcription elongation in the in vivo system or ex vivo system, as modulated by the presence of the test polydeoxyribonucleotide, as compared to the same ratio measured for a control under similar conditions; wherein in the control the linker consists essentially of one or more restriction enzyme recognition sites, or wherein in the control the linker has no substantial effect on RNA transcription termination or RNA transcription elongation, and wherein the control is otherwise substantially similar to the construct.
